(19) United States
(12) Patent Application Publication
(10) Pub. No.: US 2026/0124406 A1
(43) Pub. Date: May 7, 2026

(54) Preparation Kit for Biologic Treatments and Method for Use of Same

(71) Applicant: Quintessence Biologics Inc., Dallas, TX (US)

(72) Inventor: Sagar Nepal, Dallas, TX (US)

(73) Assignee: Quintessence Biologics Inc., Dallas, TX (US)

(21) Appl. No.: 18/938,796

(22) Filed: Nov. 6, 2024

(51) Int. Cl. A61M 11/00; A61K 47/10; A61K 47/36

(52) U.S. Cl. CPC A61M 11/007; A61K 47/10; A61K 47/36; A61M 2210/04; A61M 2210/06

(57) ABSTRACT

A preparation kit and method for reconstituting and delivering biologic treatments derived from human umbilical cord and amniotic membrane tissue. The kit includes a vial containing lyophilized biologic material, a removable seal, and a spray nozzle with a removable cap to maintain sterility. A container holds a humectant solution comprising glycerol, hyaluronic acid, or a combination thereof. In use, the humectant solution is added to the vial, the biologic material is mixed, and the spray nozzle is used for aesthetic application to human skin or hair. The vial is made of glass or medical-grade plastic to prevent contamination. The spray nozzle delivers a measured dose ranging from 0.5 mL to 5 mL. The method involves removing the seal, adding the humectant solution, attaching the spray nozzle, mixing the components, and applying the biologic via spray.

### TECHNICAL FIELD OF THE INVENTION

This invention pertains to preparation kits and methods for reconstituting and delivering biologic treatments and, in particular, this invention pertains to kits used for aesthetic applications of biologics derived from human umbilical cord and amniotic membrane tissue.

### BACKGROUND OF THE INVENTION

Biologic treatments have revolutionized the medical field by offering targeted therapies for a variety of conditions. These treatments are typically composed of large, complex molecules derived from living organisms. However, biologics often have a very limited effective life, ranging from thirty (30) minutes to two (2) hours due to their fragile nature and rapid degradation once administered or exposed to certain conditions. This short time frame presents significant challenges in ensuring these therapies are delivered effectively to patients.

One of the primary concerns with biologic treatments is maintaining their stability during transportation and storage, and then efficiently delivering the product in a timely manner to the patient. Current methods often require strict temperature controls, continuous monitoring, and immediate administration upon preparation, placing burdens on healthcare facilities and limiting access for patients who need these therapies in less controlled environments, such as at home or in remote locations. There is a clear need for improvements in the methods and systems used to deliver biologic treatments. Advancements should aim to extend the effective life of biologics, improve delivery mechanisms to ensure timely and efficient patient use, and reduce the logistical challenges currently faced by healthcare providers and patients alike.

### SUMMARY OF THE INVENTION

It would be advantageous to develop a preparation kit and method for its use that significantly improves the stability and delivery of biologic treatments for aesthetic purposes. It would be desirable to enable a biochemical-based solution that provides enhanced performance, improved usability, and extended shelf life for the biologic material. It would be further desirable to enable a convenient and effective solution that provides sterile, precise, and easily administrable doses of the biologic material, ensuring optimal results for aesthetic treatments.

Accordingly, a preparation kit and method are disclosed for reconstituting and delivering biologic treatments derived from human umbilical cord and amniotic membrane tissue. The preparation kit includes a vial containing lyophilized biologic material, a removable seal, and a spray nozzle with a removable cap to maintain sterility. A container holds a humectant solution comprising glycerol, hyaluronic acid, or a combination thereof. In use, the humectant solution is added to the vial, the biologic material is mixed, and the spray nozzle is used for aesthetic application of a biologic treatment to human skin or hair. The vial is made of glass or medical-grade plastic to prevent contamination. The spray nozzle delivers a measured dose ranging from 0.5 mL to 5 mL. In another aspect, in one embodiment, the method involves removing the seal, adding the humectant solution, attaching the spray nozzle, mixing the components, and applying the biologic treatment via spray. These and other aspects of the invention will be apparent from and elucidated with reference to the embodiments described hereinafter.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is an exploded view of one embodiment of a preparation kit for biologic treatments, illustrating key components including a vial containing lyophilized biologic material, a removable seal, a spray nozzle, and a container holding a humectant solution;

FIG. 2 is a perspective view of the fully assembled preparation kit, showing the spray nozzle attached to the vial after reconstitution of the biologic material as a biologic treatment, ready for application;

FIG. 3 is a flowchart detailing one embodiment of a method of using a preparation kit, including steps of removing a seal, adding a humectant solution, attaching a spray nozzle, mixing the components, and applying the biologic treatment; and

FIG. 4 illustrates some embodiments of an application of a reconstituted biologic material, a biologic treatment, via the spray nozzle to a target area, such as human skin or hair, for aesthetic treatments.

### DETAILED DESCRIPTION OF THE INVENTION

While the making and using of various embodiments of the present invention are discussed in detail below, it should be appreciated that the present invention provides many applicable inventive concepts, which can be embodied in a wide variety of specific contexts. The specific embodiments discussed herein are merely illustrative of specific ways to make and use the invention, and do not delimit the scope of the present invention.

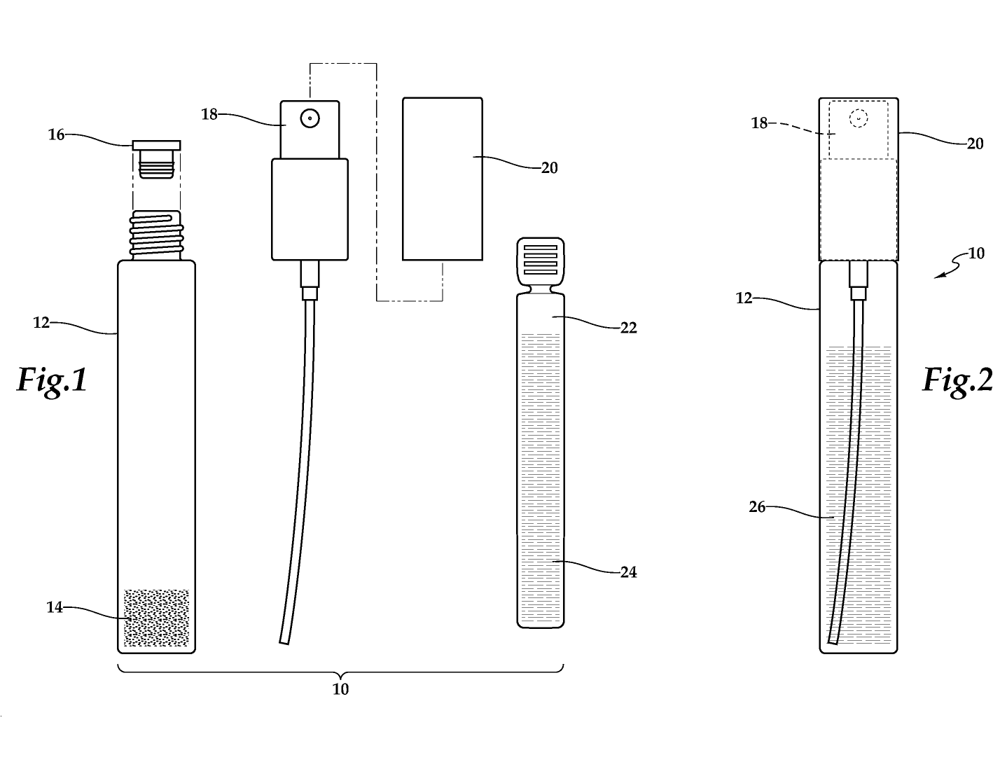

Referring initially to FIG. 1, therein is depicted one embodiment of a preparation kit for biologic treatments, which is schematically illustrated and designated 10. At the core of the preparation kit 10 is a vial 12 that contains at least one lyophilized biologic material 14. In one embodiment, the lyophilized biologic material is derived from at least one of human umbilical cord and amniotic membrane tissue, each of which provides therapeutic benefits for skin and hair treatments. The lyophilization process preserves the lyophilized biologic material by removing moisture, allowing for long-term storage without compromising its efficacy. The vial 12 may be made of a material such as glass or medical-grade plastic to ensure compatibility with the biologic material and prevent contamination. This selection of materials ensures the vial 12 is durable and maintains the sterility of the biologic material over time. The vial 12 is preferably made of borosilicate glass, as it offers superior compatibility with biologic materials and minimizes risks of degradation or contamination compared to other materials.

The vial 12 may be sealed by a removable seal 16, which is designed to protect the contents of the vial 12 and maintain a sterile environment until the preparation kit 10 is ready for use. The removable seal 16 ensures that the lyophilized biologic material 14 remains stable during transportation and storage. When the preparation process begins, the removable seal 16 is removed, allowing the next steps of the procedure to take place. A spray nozzle 18 is configured to attach securely to the vial 12 once the preparation process is complete. The spray nozzle 18 plays a crucial role in delivering the reconstituted biologic material in a precise and controlled manner. The spray nozzle 18 includes a removable cap 20, which maintains sterility of the spray mechanism before use. This removable cap 20 ensures that no contaminants reach the nozzle, preserving the sterility of the biologic solution during its preparation. Once the removable cap 20 is removed, the spray nozzle 18 is ready for use, providing a measured dose of the reconstituted biologic material with each spray.

In the preparation kit 10, there is also a container 22 that holds a humectant solution 24, which is a key component of the reconstitution process and is formulated to be one of either a glycerol-based material or a hyaluronic acid-based material. Both glycerol and hyaluronic acid are humectants that attract and retain moisture, which is essential for extending the effective time of the biologic material once applied to the skin or hair. The humectant solution 24 serves to rehydrate the lyophilized biologic material 14, transforming it from its powder form into a liquid form that can be easily applied via the spray nozzle 18. During the preparation process, the removable seal 16 is removed, and the humectant solution 24 is added to the vial 12. The lyophilized biologic material 14 and the humectant solution 24 are then mixed within the vial 12 to reconstitute the biologic material into a form suitable for application; namely, a reconstituted biologic solution 26. After mixing, the spray nozzle 18 is attached to the vial 12, and the preparation kit 10 is ready for use.

The lyophilized biologic material 14 maintains stability for up to twenty-four (24) months when stored at room temperature due to the moisture removal during lyophilization. When stored under refrigeration at 2°C to 8°C, its stability can extend to thirty-six (36) months, further minimizing degradation processes at lower temperatures. Before reconstitution, the vial 12 containing the lyophilized biologic material 14 should be stored at room temperature (15°C to 25°C) in a dry location, protected from direct sunlight and moisture, which preserves stability without refrigeration. The container 22 should also be stored at room temperature, shielded from extreme temperatures and direct light. After reconstitution, the reconstituted biologic material as the biologic treatment 26 should ideally be used immediately; if delayed, refrigeration at 2°C to 8°C is recommended, and use within seventy-two (72) hours is advised, with the reconstituted biologic treatment 26 protected from light to prevent degradation.

The lyophilized biologic material 14 rapidly dissolves in the hydrophilic humectant solution 24, leveraging glycerol and hyaluronic acid’s properties to stabilize the biologic proteins and prevent aggregation. Moisture retention from these humectants enhances skin hydration, potentially increasing biologic absorption and treatment efficacy. A specific ratio of lyophilized powder to humectant solution 24 is critical; 1.5 mL of humectant solution added to a precise amount of biologic material 14 as a powder ensures optimal reconstitution and stability. Once reconstituted, the biologic treatment 26 should ideally be applied immediately. If not used right away, the biologic treatment 26 should be stored refrigerated at 2°C to 8°C and protected from light, with application recommended within seventy-two (72) hours to maintain therapeutic efficacy.

In FIG. 1, the individual components of the preparation kit 10 are shown separately to provide a clear understanding of how the preparation kit 10 is assembled and used. Each component—the vial 12, the lyophilized biologic material 14, the removable seal 16, the spray nozzle 18, the removable cap 20, the container 22, and the humectant solution 24—is essential to the function of the preparation kit 10, ensuring that the reconstituted biologic solution as the biologic treatment 26 is safely stored, reconstituted, and applied with precision.

FIG. 2 illustrates a perspective view of the fully assembled preparation kit 10, demonstrating how the components fit together after the preparation process is completed. As illustrated, the vial 12 with the spray nozzle 18 securely attached and the preparation kit 10 ready for use. Once the preparation process begins, the removable seal 16 is taken off, exposing the lyophilized biologic material 14 inside the vial 12. The next step involves adding the humectant solution 24, contained in the container 22, to the vial 12. As shown in FIG. 2, after the humectant solution 24 is added, the spray nozzle 18 is attached to the vial 12. The attachment of the spray nozzle 18 marks the final step of the assembly process, preparing the preparation kit 10 for immediate use.

The spray nozzle 18 is designed to deliver a measured dose of the reconstituted biologic material 14 with each spray. In FIG. 2, the spray nozzle 18 is shown in its fully assembled state, with the removable cap 20 in place to maintain the sterility of the spray mechanism. This removable cap 20 ensures that the spray nozzle 18 remains free from contamination until the biologic material is ready for application. The vial 12, now containing the reconstituted biologic material 14 mixed with the humectant solution 24, serves as a convenient and sterile storage unit for the biologic treatment 26. The spray nozzle 18, configured to attach securely to the top of the vial 12, ensures that the biologic treatment 26 can be delivered with precision and control. The user can remove the cap 20, press the spray nozzle 18, and administer the biologic treatment 26 to the desired area. The assembled preparation kit 10, as depicted in FIG. 2, is designed for ease of use and efficiency. As shown in some embodiments, the vial 12 is made of a material such as glass or medical-grade plastic, ensuring compatibility with the biologic material and preventing contamination.

The spray nozzle 18 is engineered to produce a fine mist suitable for uniform application on skin or hair, enhancing absorption and distribution. This mist configuration ensures effective and even coverage, supporting optimal treatment results. The preparation kit 10 is designed for single-use to maintain sterility. Once the spray nozzle 18 is attached and the removable cap 20 removed, the entire system is intended for one-time application, ensuring no cross-contamination or risk of degradation from reuse.

This figure also emphasizes the importance of the connection between the spray nozzle 18 and the vial 12, which must be airtight and secure to ensure proper functioning of the kit. Additionally, FIG. 2 highlights the compact and portable nature of the assembled preparation kit 10. Its design allows for ease of transport and storage, making it convenient for both clinical and at-home use. The ability to store the preparation kit 10 in a sterile, sealed form until it is needed ensures that the biologic material remains effective and safe for application. In summary, FIG. 2 provides a clear depiction of how the individual components—vial 12, spray nozzle 18, removable cap 20, and the biologic material 14—come together to form the fully assembled preparation kit 10. The preparation kit 10 may contain a total of 1.5 mL of reconstituted biologic solution, allowing the user to control application needs based on the treatment area. The spray nozzle does not include a separate dosing mechanism. This figure emphasizes the functional relationship between the components and the ease with which the kit can be assembled and used to deliver biologic treatments for aesthetic purposes.

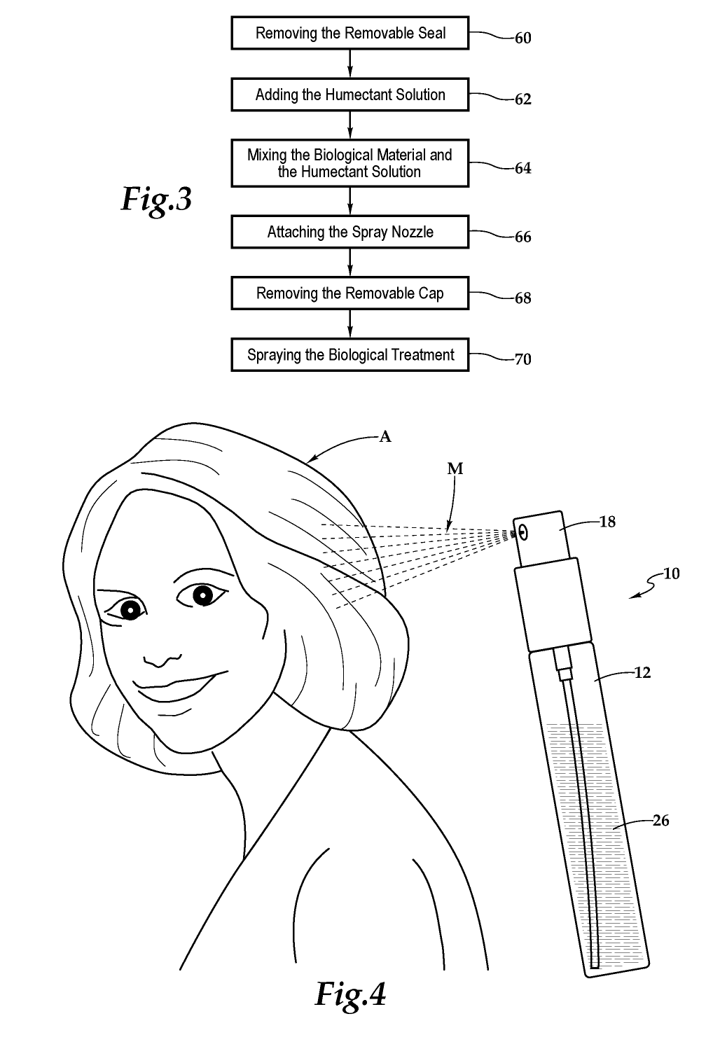

FIG. 3 illustrates a flowchart detailing one embodiment of a methodology using the preparation kit 10 for biologic treatments. The process begins with preparing the biologic material and concludes with its application to human skin or hair. The method starts at block 60, which is entitled, “Removing the Removable Seal.” The user begins by removing the seal 16 from the top of the vial 12. This seal 16 protects the lyophilized biologic material 14 from contamination during storage and transportation, ensuring that it remains sterile. Once the removable seal 16 is removed, the vial 12 is exposed, ready to receive the humectant solution 24.

Next is block 62, which is entitled, “Adding the Humectant Solution.” The humectant solution, stored in the container 22, is carefully added to the vial 12, which contains the lyophilized biologic material 14. The humectant solution 24 may be glycerol-based, hyaluronic acid-based, or a combination of both. This solution is essential for rehydrating the lyophilized biologic material, allowing it to transition into a liquid form and the biologic treatment 26. At block 64, which is entitled, “Mixing the Biologic Material and the Humectant Solution,” discusses that after the humectant solution is added, the user gently agitates the vial 12 to mix the contents. This ensures the lyophilized biologic material 14 fully dissolves in the humectant solution 24, reconstituting it into a liquid suitable for application as the biologic treatment 26.

In block 66: “Attaching the Spray Nozzle”, the spray nozzle 18 is securely connected to the vial 12. The spray nozzle 18 creates an airtight seal, ensuring that the biologic material 14 can be dispensed properly. The spray nozzle 18 is protected by a removable cap 20, which ensures sterility until the biologic material is ready for use. Next, block 68: “Removing the Removable Cap” is performed. The user removes the removable cap 20 from the spray nozzle 18, exposing the spray nozzle 18 for application. This step is crucial for maintaining the sterility of the nozzle up until the moment of application. Finally, block 70: “Spraying the Biologic Treatment” involves the application of the reconstituted biologic material as the biologic treatment 26. The user presses the spray nozzle 18 to release a measured dose of the biologic treatment 26. The spray is applied directly to the desired area, such as human skin or hair, for aesthetic purposes. The precision of the spray nozzle 18 ensures that the biologic material is applied in a consistent and controlled manner. FIG. 3 presents the entire process in a structured, step-by-step manner, ensuring clarity for users. Each block corresponds to a specific action in the method, from preparation to application, making the kit easy to use and effective in delivering the biologic treatment.

FIG. 4 illustrates the final step in using the preparation kit 10, showing the application of the reconstituted biologic material as the biologic treatment 26 to human skin or hair via the spray nozzle 18. In this figure, the biologic material 14, which has been reconstituted with the humectant solution 24, is dispensed as a fine mist M directly onto the treatment area A. Once the spray nozzle 18 has been securely attached to the vial 12, and the removable cap 20 has been taken off, the preparation kit 10 is ready for use. The user applies pressure to the spray nozzle 18, releasing a measured dose of the biologic treatment 26. This allows for precise and controlled application, ensuring that the biologic treatment 26 is evenly distributed across the target area.

The reconstituted biologic material 14, combined with the humectant solution 24, is formulated as the biologic treatment 26 to enhance moisture retention, which is particularly beneficial for skin and hair treatments. As the spray is applied, the humectant solution works to hydrate the area, helping the biologic material remain effective for an extended period. This figure highlights the key advantage of using humectants like glycerol or hyaluronic acid, which aid in moisture retention and improve the efficacy of the biologic treatment.

Additionally, FIG. 4 demonstrates the ease of use of the spray nozzle 18, which is designed to dispense the biologic treatment 26 in a controlled manner, ensuring consistency in dosage and application. The user-friendly design of the kit allows for straightforward administration, making it suitable for both clinical and at-home use. The spray nozzle 18 ensures that each dose is accurate, providing consistent results with each application. This figure also emphasizes the sterile and controlled nature of the application process. By keeping the spray nozzle 18 protected with a removable cap 20 before use, and ensuring the vial 12 remains sealed until reconstitution, the preparation kit 10 maintains a high level of sterility throughout the procedure. This prevents contamination and preserves the integrity of the biologic treatment 26 during storage, preparation, and application. In summary, FIG. 4 depicts the practical functionality of the preparation kit 10 in action. The biologic material 14, once reconstituted with the humectant solution 24, as the biologic treatment 26 is applied directly to the target area via the spray nozzle 18, delivering the treatment effectively and efficiently. The preparation kit 10 is particularly effective as a post-treatment for aesthetic procedures, including CO₂ laser resurfacing, microneedling, microdermabrasion, and chemical peels, reducing downtime and enhancing results. It may also be tailored for other treatments, such as wound healing and anti-aging applications.

The order of execution or performance of the methods and data flows illustrated and described herein is not essential, unless otherwise specified. That is, elements of the methods and data flows may be performed in any order, unless otherwise specified, and that the methods may include more or less elements than those disclosed herein. For example, it is contemplated that executing or performing a particular element before, contemporaneously with, or after another element are all possible sequences of execution.

While this invention has been described with reference to illustrative embodiments, this description is not intended to be construed in a limiting sense. Various modifications and combinations of the illustrative embodiments as well as other embodiments of the invention, will be apparent to persons skilled in the art upon reference to the description. It is, therefore, intended that the appended claims encompass any such modifications or embodiments.

What is claimed is:

1. A preparation kit for biologic treatments, comprising:
a vial containing at least one lyophilized biologic material derived from at least one of human umbilical cord and amniotic membrane tissue;
a removable seal configured to close the vial;
a spray nozzle attachable to the vial;
a container holding a humectant solution, the humectant solution being one of a glycerol-based material and a hyaluronic acid-based material;
wherein, in use, the removable seal is removed, the liquid material is added to the vial, the spray nozzle is attached, and the lyophilized biologic material and the humectant solution are mixed to reconstitute a biologic for aesthetic application by way of the spray nozzle to one of human skin and human hair.

2. The preparation kit as recited in claim 1, wherein the spray nozzle further comprises a removable cap for maintaining sterility prior to use.

3. The preparation kit as recited in claim 1, wherein the humectant solution added to the vial is a combination of glycerol and hyaluronic acid.

4. The preparation kit as recited in claim 1, wherein the vial is made of a material selected from the group consisting of glass and medical-grade plastic to ensure compatibility with the biologic and prevent contamination.

5. The preparation kit as recited in claim 1, wherein the vial is made of borosilicate glass to ensure compatibility with the biologic material and prevent contamination

6. The preparation kit as recited in claim 1, wherein the spray nozzle is configured to deliver a measured dose of the biologic material, wherein the dose ranges from 0.5 mL to 5 mL per spray.

7. The preparation kit as recited in claim 1, wherein the biologic material is configured for post-treatment applications in aesthetic procedures, including CO₂ laser resurfacing, microneedling, microdermabrasion, and chemical peels, to reduce downtime and enhance procedural outcomes.

8. The preparation kit as recited in claim 1, wherein the humectant solution, comprising glycerol or hyaluronic acid, enhances the hydrophilic properties and penetration of the biologic material for improved absorption upon application.

9. A preparation kit for biologic treatments, comprising:
a vial containing at least one lyophilized biologic material derived from at least one of human umbilical cord and amniotic membrane tissue;
a removable seal configured to close the vial;
a spray nozzle attachable to the vial, the spray nozzle further comprising a removable cap for maintaining sterility prior to use;
a container holding a humectant solution, the humectant solution being one of a glycerol-based material, a hyaluronic acid-based material, and a combination of glycerol and hyaluronic acid;
wherein the vial is made of a material selected from the group consisting of glass and medical-grade plastic to ensure compatibility with the biologic and prevent contamination;
wherein, in use, the removable seal is removed, the humectant solution is added to the vial, the spray nozzle is attached, and the lyophilized biologic material and the humectant solution are mixed to reconstitute a biologic for aesthetic application by way of the spray nozzle to one of human skin and human hair;
wherein the spray nozzle is configured to deliver a measured dose of the biologic material, wherein the dose ranges from 0.5 mL to 5 mL per spray; and
wherein the vial containing the lyophilized biologic material and the container holding the humectant solution are stored at room temperature before use, and wherein, after reconstitution, the biologic solution is ideally applied immediately or refrigerated at 2°C to 8°C and used within 72 hours.

10. A method for preparing a biologic treatment for aesthetic application to one of human skin and human hair, the method comprising:
providing a vial containing at least one lyophilized biologic material derived from at least one of human umbilical cord and amniotic membrane tissue;
removing a removable seal from the vial;
adding a humectant solution to the vial, the humectant solution being one of a glycerol-based material, a hyaluronic acid-based material, or a combination of glycerol and hyaluronic acid;
attaching a spray nozzle to the vial;
mixing the lyophilized biologic material and the humectant solution to reconstitute the biologic material; and
spraying the reconstituted biologic material through the spray nozzle for aesthetic application to one of human skin and human hair.

11. The method as recited in claim 10, further comprising the step of placing a removable cap over the spray nozzle to maintain sterility prior to use.

12. The method as recited in claim 10, wherein the humectant solution is a combination of glycerol and hyaluronic acid to improve moisture retention and extend the effective application time of the biologic material.

13. The method as recited in claim 10, wherein the vial is made of a material selected from the group consisting of glass and medical-grade plastic to ensure compatibility with the biologic material and prevent contamination.

14. The method as recited in claim 10, wherein the spray nozzle is configured to deliver a measured dose of the biologic material, wherein the dose ranges from 0.5 mL to 5 mL per spray.
